(19) United States
(12) Patent Application Publication
(10) Pub. No.: US 2022/0313777 A1
(43) Pub. Date: Oct. 6, 2022

(54) INHALED DELIVERY OF PEPTIDE MIMICS OF CXCL10 FOR TARGETED ANTI-FIBROTIC THERAPY

(71) Applicants: University of Pittsburgh - Of the Commonwealth System of Higher Education, Pittsburgh, PA (US); Tuskegee University, Tuskegee, AL (US)

(72) Inventors: Cecelia C. Yates, Pittsburgh, PA (US); Timothy E. Corcoran, Pittsburgh, PA (US); Zariel I. Johnson, Pittsburgh, PA (US); Jesse Jaynes, Tuskegee, AL (US)

(73) Assignees: University of Pittsburgh - Of the Commonwealth System of Higher Education, Pittsburgh, PA (US); Tuskegee University, Tuskegee, AL (US)

(21) Appl. No.: 17/641,955

(22) PCT Filed: Sep. 10, 2020
(86) PCT No.: PCT/US2020/050236

§ 371 Date: Mar. 10, 2022

Related U.S. Application Data
(60) Provisional application No. 62/898,151, filed on Sep. 10, 2019.

(51) Int. Cl. A61K 38/16; A61K 38/10; A61K 9/00; A61P 11/00; C07K 14/52

(52) U.S. Cl. CPC A61K 38/16; A61K 38/10; A61K 9/0078; A61K 9/0075; A61P 11/00; C07K 14/52

(57) ABSTRACT

Administration via inhalation of small peptides that mimic CXCL10 (FIBROKINE™ peptides) is described. The peptides can be administered as an aerosol, such as an aerosol with a droplet size small enough to reach lung alveoli. Use of the peptides to treat fibrosis, such as lung fibrosis, is described.

### CROSS REFERENCE TO RELATED APPLICATIONS

This application claims the benefit of U.S. Provisional Application No. 62/898,151, filed Sep. 10, 2019, which is herein incorporated by reference in its entirety.

### ACKNOWLEDGMENT OF GOVERNMENT SUPPORT

This invention was made with government support under grant number AR068317 awarded by the National Institutes of Health. The government has certain rights in the invention.

### FIELD

The present disclosure concerns inhaled delivery of in silico designed CXCL10 peptide mimics, as well as use of the peptides for the treatment of fibrosis, such as pulmonary fibrosis.

### BACKGROUND

Fibrosis is a pathological process by which thickened, scar-like tissue replaces healthy tissue. Lung fibrosis is a feature of several diseases, including idiopathic pulmonary fibrosis (IPF), systemic sclerosis (SSc), and other interstitial lung diseases. Regardless of the underlying diagnosis, ongoing fibrosis of lung tissue results in decreased oxygen uptake into the bloodstream. Symptoms rapidly progress from shortness of breath and coughing to acute respiratory failure in the majority of patients. The 5-year mortality rate for IPF patients is as high as 80% with a median survival from onset of symptoms of just 28 months. Quality of life is greatly diminished for these patients, who struggle to breathe during everyday tasks.

The only curative treatment for IPF is lung transplantation. This intervention has many drawbacks, including extensive eligibility requirements, scarcity of donor lungs, high cost, and long wait times for the approximately 1% of patients who do receive donated lungs. There are currently two drugs approved for the treatment of IPF. Approved in 2014, both Pirfenidone (Esbriet®) and Nintedanib (Ofev®) are administered orally. Both drugs demonstrated the ability to decrease progression rate in clinical trials (as measured by forced vital capacity), but do not reverse disease. One major limitation of these drugs is that they are delivered in high doses, resulting in systemic side effects and limited drug delivery to the lung. Thus, a need exists for an improved therapy for the treatment of IPF.

### SUMMARY

Methods of treating or inhibiting the development of fibrosis in a subject are provided. In some embodiments, the method includes administering to the subject by inhalation a therapeutically effective amount of a CXCL10 mimic peptide, or a therapeutically effective amount of a composition comprising a CXCL10 mimic peptide. In some examples, the composition includes multiple different CXCL10 mimic peptides, such as at least two, at least three, at least four, or at least five different CXCL20 mimic peptides.

In some embodiments, the CXCL10 mimic peptide or composition is administered as an aerosol. In some examples, the aerosol droplets have a particle size appropriate to penetrate deep into the lungs, for example a droplet size of between 1 and 5 μm in diameter. In specific examples, the peptide or composition is administered using a nebulizer, a dry powder inhaler or a metered dose inhaler.

In some embodiments, the fibrosis is fibrosis of the lung, liver, kidney, heart or skin of the subject. In some examples, the subject suffers from IPF.

The foregoing and other objects and features of the disclosure will become more apparent from the following detailed description, which proceeds with reference to the accompanying figures.

### BRIEF DESCRIPTION OF THE DRAWINGS

FIGS. 1A-1I are a series of graphs demonstrating that IP-10 derived peptides inhibit TGF-β-mediated induction and expression of pro-fibrotic mRNAs. Primary lung fibroblasts from scleroderma patients were treated with TGF-β alone, or with IP-10-1 (SEQ ID NO: 2; FIGS. 1A-1C), IP-10-2 (SEQ ID NO: 3; FIGS. 1D-1F), or IP-10-3 (SEQ ID NO: 4; FIGS. 1G-1I) peptide (10 ng/mL) in the presence or absence of TGF-β (10 ng/mL) for 24 hours and then analyzed by RT-qPCR for the fibrotic ECM markers tenascin C (FIGS. 1A, 1D, 1G), collagen 1 (FIGS. 1B, 1E, 1H) and fibronectin (FIGS. 1C, 1E, 1I). Treatment with TGF-β alone induced expression of mRNAs for tenascin C (TNC), collagen 1 (COL1A1), and fibronectin (FN1) in primary lung fibroblasts. When cells were treated with an IP-10 peptide in combination with TGF-β, the IP-10 peptides reduced or prevented induction in mRNA expression of TNC, COL1A1, and FN1.

FIG. 2 is a schematic of an exemplary study protocol for a bleomycin-induced lung fibrosis mouse model.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Aug. 26, 2020, 10.9 KB, which is incorporated by reference herein. In the accompanying sequence listing:
SEQ ID NOs: 1-4 are the amino acid sequences of CXCL10 peptides and variants.
SEQ ID NOs: 5-42 are the amino acid sequences of in silico designed CXCL10 mimic peptides.

### DETAILED DESCRIPTION

### I. Abbreviations

COL1A1 collagen 1

CXCL10 C-X-C chemokine ligand 10

CXCR3 C-X-C chemokine receptor 3

ECM extracellular matrix

FN1 fibronectin

IP-10 interferon-inducible protein 10

IPF idiopathic pulmonary fibrosis

SSc systemic sclerosis

TNC tenascin C

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Aerosol: A suspension of fine solid particles or liquid droplets in a gas (such as air).

Administration: The introduction of a composition (such as a protein or peptide) into a subject by a chosen route, such as via inhalation. In some embodiments herein, the CXCL10 mimic peptides are administered as an aerosol via inhalation (such as using a nebulizer).

Agonist: A drug or molecule (such as a peptide) that promotes the activity or function of another drug or molecule. For example, an agonist of a receptor is a molecule that enhances activity (such as signaling activity) of the receptor. The CXCL10 mimic peptides disclosed herein are agonists of CXCR3.

Conservative variants: “Conservative” amino acid substitutions are those substitutions that do not substantially affect or decrease an activity or antigenicity of a protein, such as a CXCL10 mimic peptide. For example, the peptides of any one of SEQ ID NOs: 1-42 can include at most about 1, at most about 2, at most about 3, at most about 4, at most about 5, at most about 6, at most about 7 or at most about 8 conservative substitutions, such as 1, 2, 3, 4, 5, 6, 7 or 8 conservative substitutions, such as 1 to 3, 1 to 5 or 2 to 6 conservative substitutions, and retain biological activity, such as the ability to bind CXCR3 and/or the ability to activate CXCR3. In particular examples, the peptide variants have no more than 3 conservative amino acid substitutions. Specific, non-limiting examples of a conservative substitution include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variant also includes the use of a substituted amino acid in place of an unsubstituted parent amino acid. Non-conservative substitutions are those that reduce an activity or antigenicity.

Contacting: Placement in direct physical association; includes both in solid and liquid form.

CXCL10 (C-X-C chemokine ligand 10): A chemokine of the CXC subfamily and ligand for the receptor CXCR3. Binding of CXCL10 to CXCR3 results in pleiotropic effects, including stimulation of monocytes, natural killer and T-cell migration, modulation of adhesion molecule expression, and inhibition of vessel formation. CXCL10 is also known as interferon-γ-inducible 10 kDa protein (IP-10).

CXCR3 (C-X-C chemokine receptor 3): A G protein-coupled receptor with selectivity for four chemokines, CXCL4, CXCL9, CXCL10 and CXCL11. Binding of chemokines to CXCR3 induces signaling and cellular responses that are involved in leukocyte trafficking, most notably integrin activation, cytoskeletal changes and chemotactic migration. Alternatively spliced transcript variants encoding different isoforms have been found for this gene. One of the isoforms (CXCR3-B) shows high affinity binding to chemokine CXCL4.

Fibrosis: A condition associated with the thickening and scarring of connective tissue. Often, fibrosis occurs in response to an injury, such as from a disease or condition that damages tissue. Fibrosis is an exaggerated wound healing response that when severe, can interfere with normal organ function. Fibrosis can occur in almost any tissue of the body, including in the lung (pulmonary fibrosis, cystic fibrosis, radiation-induced lung injury), liver (cirrhosis, biliary atresia), heart (arterial fibrosis, endomyocardial fibrosis, prior myocardial infarction), brain, skin (scleroderma, sclerosis), kidney, joints and intestine (Crohn's disease).

Idiopathic pulmonary fibrosis (IPF): Fibrosis of the lung having an unknown cause. IPF is a serious chronic disease affecting the tissues surrounding alveoli in the lungs. This disease is characterized by a progressive and irreversible decline in lung function.

Isolated: An “isolated” or “purified” biological component (such as a nucleic acid or peptide) has been substantially separated, produced apart from, or purified away from other biological components in the cell of the organism in which the component occurs, such as other chromosomal and extra-chromosomal DNA and RNA, and proteins. Nucleic acids, peptides and proteins that have been “isolated” or “purified” thus include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity, and can include protein, peptide, or nucleic acid molecules that are at least 50% isolated, such as at least 75%, 80%, 90%, 95%, 98%, 99%, or even 99.9% isolated.

Nebulizer: A device for converting a therapeutic agent (such as a peptide) in liquid form into a mist or fine spray (an aerosol) that can be inhaled into the respiratory system, such as the lungs. A nebulizer is also known as an “atomizer ” In some embodiments of the present disclosure, the nebulizer is an AEROECLIPSE® II Breath Actuated Nebulizer (BAN), an AirLife Sidestream nebulizer or an AEROGEN® Ultra vibrating mesh nebulizer.

Peptide or polypeptide: A polymer in which the monomers are amino acid residues which are joined together through amide bonds. When the amino acids are alpha-amino acids, either the

L-optical isomer or the D-optical isomer can be used, the L-isomers being preferred. The terms “polypeptide,” “peptide,” or “protein” as used herein are intended to encompass any amino acid sequence and include modified sequences such as glycoproteins. The terms “polypeptide” and “peptide” are specifically intended to cover naturally occurring proteins, as well as those which are recombinantly or synthetically produced.

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the peptides herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. For topical application to the eye, agents can be mixed, for example, with artificial tears and other emulsions.

Preventing, treating or ameliorating a disease: “Preventing” a disease refers to inhibiting the full development of a disease. “Treating” refers to a therapeutic intervention that ameliorates a sign or symptom of a disease or pathological condition (such as fibrosis) after it has begun to develop. “Ameliorating” refers to the reduction in the number or severity of signs or symptoms of a disease.

Recombinant: A recombinant protein or peptide is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques. The term “recombinant” also includes proteins and peptides that have been altered solely by addition, substitution, or deletion of a portion of the natural protein or peptide.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a particular polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. In addition, Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 90%, 95%, 96%, 97%, 98% or 99% sequence identity counted over the full length alignment with the amino acid sequence of the polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals. In some examples, the subject suffers from fibrosis.

Synthetic: Produced by artificial means in a laboratory, for example a synthetic nucleic acid or peptide can be chemically synthesized in a laboratory.

Therapeutically effective amount: A quantity of a specified agent (such as a CXCL10 mimic peptide) sufficient to achieve a desired effect in a subject, cell or culture being treated with that agent. In some embodiments, the therapeutically effective amount is the amount of peptide necessary to inhibit TGF-β signaling. In other embodiments, the therapeutically effective amount is the amount of peptide sufficient to treat or inhibit fibrosis in a subject.

Transforming growth factor-β (TGF-β): A secreted, multi-functional protein that regulates cell proliferation, cellular differentiation, apoptosis and a number of other cellular functions. Many cells synthesize TGF-β and nearly all cells express receptors for TGF-β. The term “TGF-β” refers to three different protein isoforms, TGF-β1, TGF-β2 and TGF-β3, encoded by the genes TGFB1, TGFB2, TGFB3, respectively.

### III. Overview of Several Embodiments

Described herein are small peptides that mimic CXCL10, a potent anti-fibrotic chemokine, to inhibit and reverse the pathogenesis of pulmonary fibrosis through multiple mechanisms. The disclosed peptides simultaneously target two mechanisms that contribute to fibrosis: (1) activity of pro-fibrotic TGFβ and (2) increased angiogenesis. Full-length CXCL10, through its receptor CXCR3, has potent anti-angiogenic effects in multiple tissues. In addition, CXCL10 inhibits the effects of TGFβ, a factor considered to be the major contributor to fibrosis. TGFβ increases production of extracellular matrix components, tenascin, and alpha smooth muscle actin by fibroblast cells. Full-length CXCL10 inhibits these effects of TGFβ.

An adaptive algorithm was used for in silico prediction-based functional peptide design to identify multiple small peptides that mimic the functions of full-length CXCL10. The disclosed peptides were specifically designed to act through CXCR3 and thereby function as CXCL10 mimics to inhibit the effects of TGFβ, and to halt angiogenesis and excessive tissue remodeling. To direct delivery of the peptides to the lung, decrease systemic side effects, and increase effectiveness of the anti-fibrotic CXCL10 mimic (FIBROKINE™) peptides, an innovative strategy was developed to deliver the peptides through an inhaled mechanism. The adult lung has a high absorptive capacity due to its large surface area. Based on animal and clinical models, the effective dose of an inhaled drug is typically 10- to 100-fold lower than an oral form. Furthermore, inhaled drugs are not subject to first-pass metabolism and are less likely to cause liver damage.

There are currently no approved drugs that have been demonstrated to reverse lung fibrosis. The only curative treatment for lung fibrosis is lung transplantation, which is limited by the number of donor lungs and eligibility of potential recipients. Other treatments for lung fibrosis include oxygen therapy, use of steroids, and palliative care for advanced disease. In 2014, two orally delivered drugs, Pirfenidone and Nintedanib, were approved for the treatment of idiopathic pulmonary fibrosis (IPF). These drugs slow progression of disease, but do not significantly impact long-term survival and do not diminish many symptoms of disease. These medications are also not effective in all patients. The mechanism of action of Pirfenidone is not completely understood, but it is known to inhibit the production and activity of TGFβ and can diminish inflammation. Nintedanib acts by inhibiting growth factor receptors involved in the pathogenesis of fibrosis. Unfortunately, about 20% of patients have to cease treatment due to side effects including gastrointestinal upset, nausea, skin rashes, and liver toxicity.

CXCL10, a naturally expressed chemokine, inhibits the pathogenesis of fibrosis by at least two mechanisms. Acting through its receptor CXCR3, CXCL10 inhibits activity of the pro-fibrotic growth factor TGFβ and it stops angiogenesis. The CXCL10 peptide mimics disclosed herein are designed to act as agonists for CXCR3, similar to CXCL10. In some embodiments, the CXCL10 mimic peptides are delivered through an optimized inhalation mechanism to target them directly to the lung. The peptides may be aerosolized or delivered in powder form or other inhaled forms capable of penetrating into the deep lung area.

Provided herein is a method of treating or inhibiting the development of fibrosis in a subject. In some embodiments, the method includes administering to the subject by inhalation a therapeutically effective amount of a CXCL10 mimic peptide, or a therapeutically effective amount of a composition comprising a CXCL10 mimic peptide.

In some embodiments, the CXCL10 mimic peptide is 12 to 30 amino acids in length, such as 12 to 25, 13 to 17 or 14 to 16 amino acids in length. In some examples, the peptide is 12, 13, 14, 15, 16, 17, 18, 19, 20, 21, 22, 23, 24, 25, 26, 27, 28, 29 or 30 amino acids in length.

In some embodiments, the amino acid sequence of the CXCL10 mimic peptide has 1, 2, 3, 4, 5, 6, 7 or 8 conservative amino acid substitutions relative to any one of SEQ ID NOs: 1-42. In some examples, the amino acid sequence of the CXCL10 mimic peptide has no more than 3, no more than 2 or no more than 1 conservative amino acid substitution relative to any one of SEQ ID NOs: 1-42.

In some embodiments, the amino acid sequence of the CXCL10 mimic peptide is at least 90%, at least 95%, at least 96%, at least 97%, at least 98% or at least 99% identical to any one of SEQ ID NOs: 1-42. In some examples, the amino acid sequence of the CXCL10 mimic peptide comprises or consists of any one of SEQ ID NOs: 1-42.

In some embodiments, the composition includes at least two, at least three, at least four, at least five, at least six, at least seven or at least 8 different CXCL10 mimic peptides. For example, the composition can include 1 to 8 peptides, 2 to 7 peptides, 3 to 6 peptides, or 4 to 5 peptides.

In some embodiments, the composition further includes a pharmaceutically acceptable carrier.

In some embodiments, the peptide or composition is administered as an aerosol. In some examples, the aerosol droplets are between about 1 μm and about 5 μm in diameter, for example about 2 to about 4 μm in diameter. In the context of the present disclosure “about 1 μm” includes 0.95 to 1.05 μm and “about 5 μm” includes 4.95 to 5.05 μm. In other examples, the aerosol droplets are less than or equal to 3 μm in diameter, such as about 3 μm, about 2.5 μm, about 2 μm, about 1.5 μm, or about 1 μm in diameter. In other examples, the aerosol droplets are less than or equal to 8 μm in diameter, such as about 8 μm about 7.5 μm, about 7 μm, about 6.5 μm, about 6 μm, about 5.5 μm, about 5 μm, about 4.5 μm, about 4 μm, or about 3.5 μm.

In some embodiments, the peptide or composition is administered using a nebulizer. Any nebulizer capable of converting the peptide or composition into an aerosol with an appropriate droplet size for delivery to the lung can be used. In some examples, the nebulizer is an AEROECLIPSE® II Breath Actuated Nebulizer (BAN), an AirLife Sidestream nebulizer or an AEROGEN® Ultra vibrating mesh nebulizer. In other embodiments, the peptide or composition is administered using a dry powder inhaler or a metered dose inhaler.

In some embodiments in which a composition containing one or more peptides is delivered to the subject, the composition includes about 50 ng/ml to about 1000 ng/ml of peptide, such as about 100 ng/ml to about 500 ng/ml, about 150 ng/ml to about 450 ng/ml, about 200 ng/ml to about 400 ng/ml, or about 250 ng/ml to about 350 ng/ml of peptide.

In some embodiments, the peptide includes at least one chemical modification. In some examples, the peptide includes polyethylene glycol (PEG), one or more D-amino acids (d-AA), N-acetylation, lipidization, or B12 conjugation. In other examples, the peptide is cyclized.

In some embodiments, the fibrosis is fibrosis of lung, liver, kidney, heart or skin of the subject. In some example when the fibrosis is lung fibrosis, the subject suffers from idiopathic pulmonary fibrosis (IPF). In some examples when the fibrosis is cardiac fibrosis, the subject has suffered from a myocardial infarction. In some examples wherein the fibrosis is skin fibrosis, the subject has systemic sclerosis (SSc).

### IV. Methods of Treating and Inhibiting the Development of Fibrosis

Fibrosis is a pathological process by which scar-like tissue replaces healthy tissue. Organ fibrosis can occur in localized diseases, such as idiopathic pulmonary fibrosis (IPF) or in systemic diseases like systemic sclerosis (SSc). When fibrosis takes place in the lung, the resulting tissue is suboptimal for transporting oxygen into the bloodstream, resulting in symptoms that progress from shortness of breath to acute respiratory failure in patients with IPF. The prognosis for IPF patients is dismal, with a 60-80% 5-year mortality rate. Currently, there are no approved therapeutics for IPF that reverse fibrosis. As a result, IPF remains a significant burden on the healthcare system, ranking as the most common indication for lung transplantation in the United States. Similarly, fibrosis of the skin in SSc and other disorders leads to stiffened skin, pain and decreased quality of life for patients.

There are no currently approved drugs that have been shown to reverse lung or skin fibrosis. Thus, the only curative treatment for pulmonary fibrosis is lung transplantation—an expensive approach that is limited by both the number of available donor lungs and recipient eligibility. Other treatment options include oxygen therapy, use of steroids, and palliative care for advanced disease. Two recently approved drugs, Pirfenidone and Nintedanib, were shown to modestly decrease rate of progression in IPF. However, these drugs were not effective in some patient subsets.

Current treatment for dermal fibrosis includes injection of corticosteroids into areas of fibrosis (hypertrophic scars and keloids) and surgical removal. Steroid injections cause atrophy of surrounding tissue, including fat and muscle, and keloids frequently recur following surgical resection. In SSc patients, immunosuppressive agents are used with low efficacy.

CXCR3 and its natural ligands inhibit the pathogenesis of fibrosis by at least two mechanisms. Acting through its receptor CXCR3, CXCL10 inhibits activity of the pro-fibrotic growth factor TGFβ and stops angiogenesis. The peptides disclosed herein are designed to act as agonists for CXCR3 in a similar fashion to these endogenous ligands.

CXCL10, also known as interferon gamma-induced protein 10 (IP-10), is an inhibitor of fibroblast function. CXCL10 is secreted by several cell types, including fibroblasts. Studies have demonstrated the critical role of CXCL10 in the post-MI remodeling response using CXCL10-/-mice. In these studies, the lack of CXCL10 resulted in significantly more infiltrating leukocytes, macrophages, and αSMA-expressing myofibroblasts into the infarct. This increased cellular infiltration promoted a larger infarction and dilation at day 7 and significantly more systolic dysfunction at day 28. CXCL10 was found to inhibit basic fibroblast growth factor (bFGF)-induced migration of fibroblasts but did not appear to alter fibroblast proliferation or apoptosis. Subsequent studies identified that the CXCL10 actions in the infarcted heart and isolated cardiac fibroblasts were mediated through proteoglycans. Together these studies indicate a protective role for CXCL10, which includes the attenuation of cardiac fibroblast activation and collagen secretion. In addition, CXCL10 preserved cardiac function during the post-MI remodeling process.

A CXCL10 peptide consisting of the α-helical domain (residues 77-98 of human CXCL10; SEQ ID NO: 1) that mimics the action of CXCL10 on dermal endothelial cells via CXCR3 has been described (PCT Publication Nos. WO 2013/032853 and WO 2015/112505, incorporated herein by reference). Specifically, this peptide, referred to as IP-10p, activates the CXCR3B isoform that inhibits migration and proliferation in various cell types. In the heart, however, evidence suggests that CXCL10 signals through an alternative CXCR3-independent pathway to reduce fibrosis after MI. Although IP-10p has been shown to be able to inhibit angiogenesis, it has not been tested in anti-fibrotic applications.

Small peptides that act on the chemokine receptor CXCR3 were designed in silico. Naturally occurring ligands of CXCR3, such as CXCL10 and CXCL 11, are potent anti-fibrotic chemokines. The small peptides were designed to inhibit and reverse the pathogenesis of fibrosis through multiple mechanisms. In particular, the peptides were designed to have the ability to simultaneously target two activities that contribute to fibrosis: (1) the activity of pro-fibrotic TGFβ and (2) increased angiogenesis. Activation of CXCR3 by natural ligands has potent anti-angiogenic effects in multiple tissues. In addition, it has been determined that CXCL10 inhibits the effects of TGFβ, a factor considered to be a major contributor to fibrosis. TGFβ increases production of extracellular matrix components, tenascin, and alpha smooth muscle actin by fibroblast cells. Full-length CXCL10 inhibits these effects of TGFβ.

To generate the small peptides, an adaptive algorithm for in silico prediction-based functional peptide design was used to identify multiple small peptides that mimic the functions of full-length CXCR3 ligands (see Example 1). These peptides were specifically designed to act through CXCR3, thereby inhibiting the effects of TGFβ and halting angiogenesis and excessive tissue remodeling.

One challenge to developing treatments for fibrotic diseases is the heterogeneity of patient populations. The rate of disease progression is variable and difficult to predict. This heterogeneity also results in differential response of patients to therapeutics. The disclosed peptides are all designed to work through CXCR3, but may have different magnitudes of response on angiostatic and anti-TGFβ pathways. Therefore, the disclosed peptides or combinations thereof can be tailored to individuals based on their disease need in a personalized medicine approach.

Provided herein is a method of treating or inhibiting the development of fibrosis in a subject. In some embodiments, the method includes administering to the subject by inhalation a therapeutically effective amount of a CXCL10 mimic peptide or a composition that includes the peptide.

### V. Embodiments

Embodiment 1. A method of treating or inhibiting the development of fibrosis in a subject, comprising administering to the subject by inhalation a therapeutically effective amount of a CXCL10 mimic peptide, or a therapeutically effective amount of a composition comprising a CXCL10 mimic peptide, wherein the peptide or composition is administered as an aerosol, thereby treating or inhibiting the development of fibrosis in the subject.

Embodiment 2. The method of embodiment 1, wherein the CXCL10 mimic peptide is 12 to 30 amino acids in length.

Embodiment 3. The method of embodiment 1 or embodiment 2, wherein the amino acid sequence of the CXCL10 mimic peptide is at least 90% identical to any one of SEQ ID NOs: 1-42.

Embodiment 4. The method of embodiment 3, wherein the amino acid sequence of the CXCL10 mimic peptide is at least 95% identical to any one of SEQ ID NOs: 1-42.

Embodiment 5. The method of embodiment 4, wherein the amino acid sequence of the CXCL10 mimic peptide comprises or consists of:
any one of SEQ ID NOs: 1-42;
any one of SEQ ID NOs: 1-4;
any one of SEQ ID NOs: 5-7;
any one of SEQ ID NOs: 8-9;
any one of SEQ ID NOs: 10-11;
any one of SEQ ID NOs: 12-14;
any one of SEQ ID NOs: 15-17;
any one of SEQ ID NOs: 18-19;
any one of SEQ ID NOs: 20 and 24;
any one of SEQ ID NOs: 21-23;
any one of SEQ ID NOs: 25-29;
any one of SEQ ID NOs: 30-42;
any one of SEQ ID NOs:35-37; or
any one of SEQ ID NOs: 38-42.

Embodiment 6. The method of any one of embodiments 1-5, wherein the composition comprises at least two, at least three, at least four or at least five different CXCL10 mimic peptides.

Embodiment 7. The method of any one of embodiments 1-6, wherein the composition further comprises a pharmaceutically acceptable carrier.

Embodiment 8. The method of any one of embodiments 1-7, wherein the aerosol droplets are between 1 and 5 μm in diameter.

Embodiment 9. The method of any one of embodiments 1-8, wherein the peptide or composition is administered using a nebulizer, a dry powder inhaler or a metered dose inhaler.

Embodiment 10. The method of any one of embodiments 1-9, wherein the composition comprises about 100 ng/ml to about 500 ng/ml of the peptide.

Embodiment 11. The method of any one of embodiments 1-10, wherein the fibrosis is fibrosis of lung, liver, kidney, heart or skin of the subject.

Embodiment 12. The method of embodiment 11, wherein the subject suffers from idiopathic pulmonary fibrosis (IPF).

Embodiment 13. The method of any one of embodiments 1-12, further comprising treating the subject with oxygen therapy, a steroid, or an immunosuppressive agent.

Embodiment 14. A CXCL10 mimic peptide, or a composition comprising a CXCL10 mimic peptide, for use in a method of treating or inhibiting the development of fibrosis in a subject, wherein a therapeutically effective amount of the peptide or composition is administered to the subject by inhalation as an aerosol.

Embodiment 15. The peptide or composition of embodiment 14, wherein the CXCL10 mimic peptide is 12 to 30 amino acids in length.

Embodiment 16. The peptide or composition of embodiment 14 or embodiment 15, wherein the amino acid sequence of the CXCL10 mimic peptide is at least 90% identical to any one of SEQ ID NOs: 1-42.

Embodiment 17. The peptide or composition of embodiment 16, wherein the amino acid sequence of the CXCL10 mimic peptide is at least 95% identical to any one of SEQ ID NOs: 1-42.

Embodiment 18. The peptide or composition of embodiment 17, wherein the amino acid sequence of the CXCL10 mimic peptide comprises or consists of:
any one of SEQ ID NOs: 1-42;
any one of SEQ ID NOs: 1-4;
any one of SEQ ID NOs: 5-7;
any one of SEQ ID NOs: 8-9;
any one of SEQ ID NOs: 10-11;
any one of SEQ ID NOs: 12-14;
any one of SEQ ID NOs: 15-17;
any one of SEQ ID NOs: 18-19;
any one of SEQ ID NOs: 20 and 24;
any one of SEQ ID NOs: 21-23;
any one of SEQ ID NOs: 25-29;
any one of SEQ ID NOs: 30-42;
any one of SEQ ID NOs:35-37; or
any one of SEQ ID NOs: 38-42.

Embodiment 19. The peptide or composition of any one of embodiments 14-18, wherein the composition comprises at least two, at least three, at least four or at least five different CXCL10 mimic peptides.

Embodiment 20. The peptide or composition of any one of embodiments 14-19, wherein the composition further comprises a pharmaceutically acceptable carrier.

Embodiment 21. The peptide or composition of any one of embodiments 14-20, wherein the aerosol droplets are between 1 and 5 μm in diameter.

Embodiment 22. The peptide or composition of any one of embodiments 14-21, wherein the peptide or composition is administered using a nebulizer, a dry powder inhaler or a metered dose inhaler.

Embodiment 23. The peptide or composition of any one of embodiments 14-22, wherein the composition comprises about 100 ng/ml to about 500 ng/ml of the peptide.

Embodiment 24. The peptide or composition of any one of embodiments 14-23, wherein the fibrosis is fibrosis of lung, liver, kidney, heart or skin of the subject.

Embodiment 25. The peptide or composition of embodiment 24, wherein the subject suffers from idiopathic pulmonary fibrosis (IPF).

Embodiment 26. The peptide of composition of any one of embodiments 14-25, wherein the subject is further administered oxygen therapy, a steroid, or an immunosuppressive agent.

The following examples are provided to illustrate certain particular features and/or embodiments. These examples should not be construed to limit the disclosure to the particular features or embodiments described.

### EXAMPLES

### Example 1: CXCL10 Mimic (FIBROKINE™) Peptides

This example provides the sequences of 42 small peptides that function as CXCL10 mimics Four of the peptides are CXCL10 (also known as “IP-10”) peptides or variants thereof (SEQ ID NOs: 1-4). In addition, a series of small peptides (13 to 25 amino acids in length) were developed by in silico prediction-based functional peptide design to mimic CXCL10 (SEQ ID NOs: 5-42). The amino acid sequences of each peptide are provided in Table 1.

| Name | Peptide | SEQ ID NO: |
|---|---|---|
| IP-10p | PESKAIKNLLKAVSKEMSKRSP | 1 |
| IP-10-1 | PESKVIKNLLKVASKERSKRSP | 2 |
| IP-10-2 | PESRVIKNLLRVVSKEKSRRSP | 3 |
| IP-10-3 | FAKAIKNFAKAVAKFA | 4 |
| CXCL10-1 | VRSTSISGGSISTSRV | 5 |
| CXCL10-2 | IIPASQFGGFPQSAPII | 6 |
| CXCL10-3 | FSPRVEIGGIEVRPSF | 7 |
| CXCL11-1 | FPMFKGGKFMPF | 8 |
| CXCL11-2 | RLSLSIGGISLSR | 9 |
| CXCR3LP2-1 | DDHKEINAGGANIEKHD | 10 |
| CXCR3LP2-2 | DDHKEIGGIEKHD | 11 |
| CXCL10-3-1 | FSPHVEIGGIEVHPSF | 12 |
| CXCL10-3-2 | FTPHVEIGGIEVHPTF | 13 |
| CXCL10-3-3 | FTHVEIGGIEVHTF | 14 |
| CXCL10-1MI | SISTSRVGGVRSTSIS | 15 |
| CXCL10-2MI | FPQSAPIGGIIPASQF | 16 |
| CXCL10-3MI | IEVRPSFGGFSPRVEI | 17 |
| CXCL11-1MI | KFMPFGGFPMFK | 18 |
| CXCL11-2MI | ISLSRGGRLSLSI | 19 |
| CXCR3LP2-1MI | ANIEKHDGGDDHKEINA | 20 |
| CXCL10-3-1MI | IEVHPSFGGFSPHVEI | 21 |
| CXCL10-3-2MI | IEVHPTFGGFTPHVEI | 22 |
| CXCL10-3-3MI | IEVHTFGGFTHVEI | 23 |
| CXCR3LP2-2MI | EKHDIGGDDHKEI | 24 |
| CR3-1 | DRFNSFRQPGGQRFSNFRD | 25 |
| CR3-2 | FVWQVAARVGGVRAAVQWVF | 26 |
| CR3-3 | DGVQPFNYQGGQYNFPQVGD | 27 |
| CRF-4 | NDALAGLRMLIRGGRIMRLGALADN | 28 |
| CRF-5 | LRMLIRGGRILMRL | 29 |
| CR3-1-1 | DRFNSYRQPGGQRYSNFRD | 30 |
| CR3-1-2 | DRYNSYRQPGGQRYSNYRD | 31 |
| CR3-2-1 | FVWQVFARVGGVRAFVQWVF | 32 |
| CR3-2-2 | FVWFARYGGYRAFWVF | 33 |
| CR3-3-1 | DGFQPFNYQGGQYNFPQFGD | 34 |
| CRF-4-1 | NDALFGLRMLIRGGRIMRLGFLADN | 35 |
| CRF-4-2 | NDWLFRMLIRGGRIMRFLWDN | 36 |
| CRF-4-3 | WLFRMWIRGGRWMRFLW | 37 |
| CRF-5-1 | LRFLIRGGRILFRL | 38 |
| CRF-5-2 | LRFLFRGGRFLFRL | 39 |
| CRF-5-3 | FRFLFRGGRFLFRF | 40 |
| CRF-5-4 | FRWLFRGGRFLWRF | 41 |
| CRF-5-5 | FRWLWRGGRWLWRF | 42 |

To identify lead candidates, the effect of the CXCL10 peptide mimics on fibroblast cell activity are assessed, such as by evaluating fibroblast migration, myofibroblast differentiation, and survival in vitro. Additionally, these peptides are tested in a preclinical animal model of lung fibrosis to determine their ability to slow and/or halt the progression of fibrotic extracellular matrix (ECM) deposition by radiologic and histopathologic assessment using standard lung fibrosis diagnostic criteria.

Studies are also conducted to optimize peptide delivery parameters to reach the lung alveoli. Peptides delivered as an aerosol spray must generally be between about 1 and about 5 μm in diameter to reach the lung alveoli, the site of fibrosis in IPF patients. Parameters of aerosolization are determined, and peptides subjected to this process are tested to ensure their bioactivity is unaffected by this process. In vitro, CXCR3 binding affinity and TGFβ-induced fibroblast functional studies are performed to ensure the peptides are not adversely affected by this process

Follow-on studies are performed to establish the effect of candidate peptides on the extent of fibrosis in a mouse model of bleomycin-induced lung fibrosis. Peptide efficacy after treatment of bleomycin over a two-month time course post-dosing is evaluated to characterize changes in lung inflammation, lung function, collagen deposition, pathology and clinically validated serum ECM biomarkers. These markers are associated with the diagnosis and prognosis of human IPF.

### Example 2: Ex Vivo Cotoxicity and Efficacy Studies

Studies are performed using well-established assays to compare activity and performance of at least three candidate peptides. Briefly, studies evaluate binding between the candidate peptides and the CXCR3 receptor, fibroblast and endothelial cell viability, inhibition of fibrotic effects of TGFβ, and angiostatic properties of the peptides at various doses. This information will inform in vivo studies.

### Example 3: Effect Of CXCL10 Mimic Peptides on Expression of Pro-Fibrotic mRNA

This example describes inhibition of expression of pro-fibrotic mRNAs by CXCL10 mimic peptides in primary lung fibroblasts from patients with scleroderma.

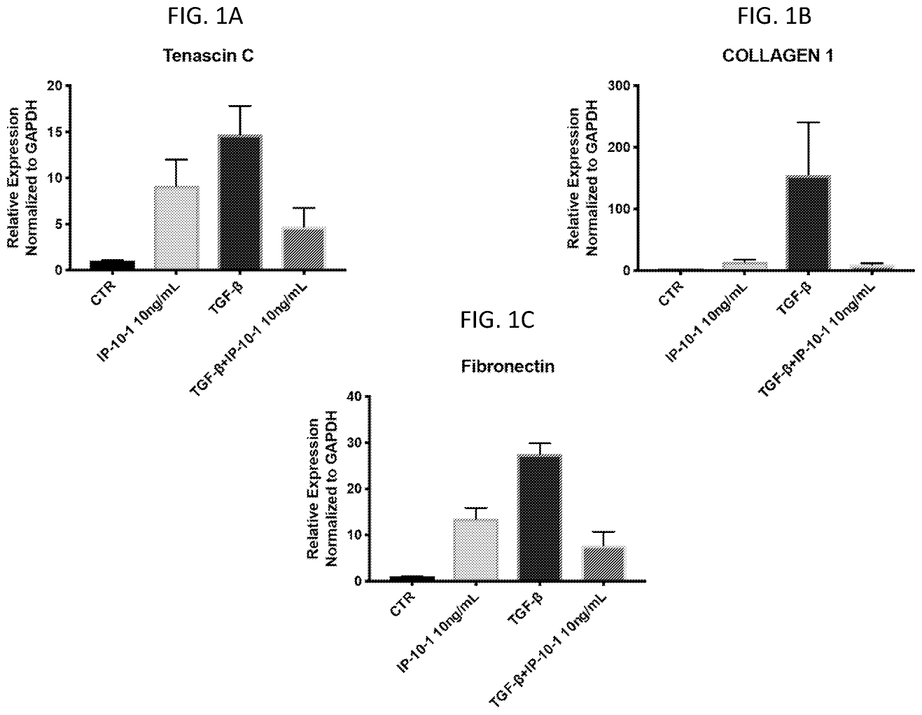

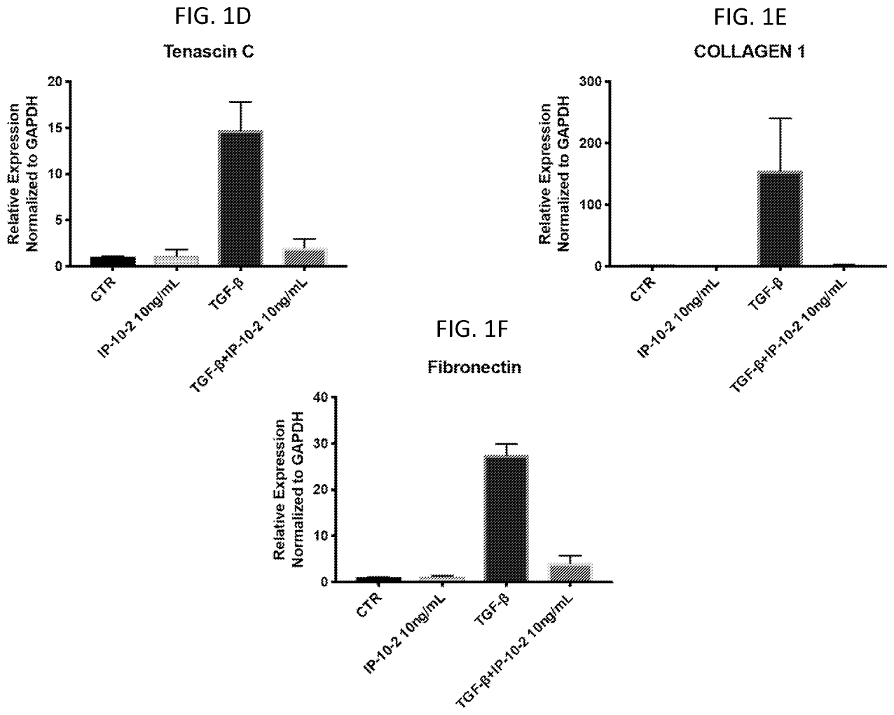

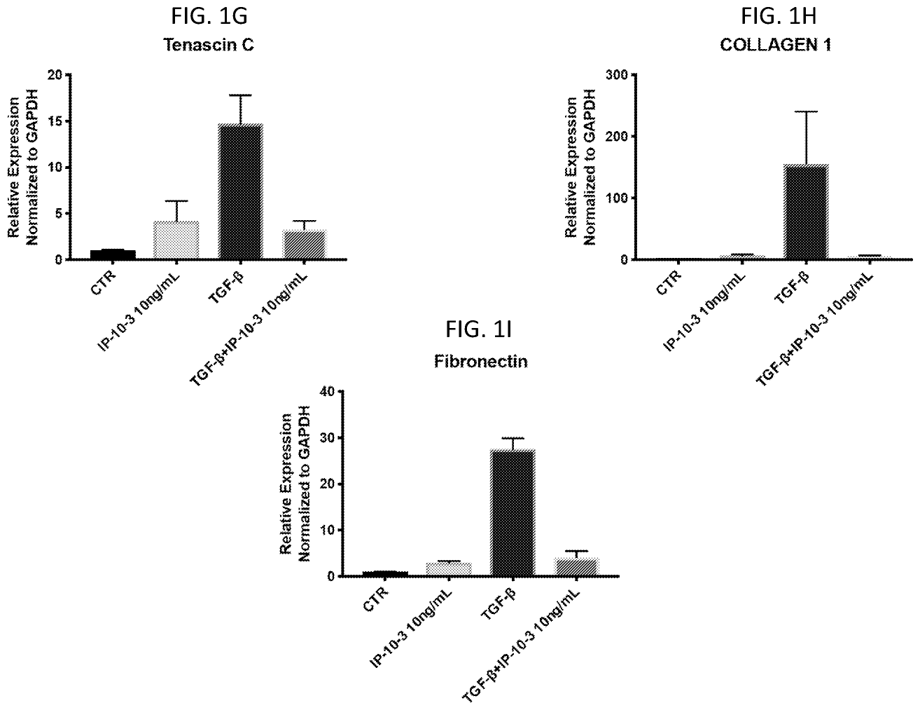

Primary lung fibroblasts from scleroderma patients were plated in 6-well plates in complete medium and incubated overnight. The next day, the medium was changed to quiescent medium and the cells were incubated in this medium for 24 hours. The following day, cells were left untreated, treated with 10 ng/mL of IP-10-1 (SEQ ID NO: 2), IP-10-2 (SEQ ID NO: 3) or IP-10-3 (SEQ ID NO: 4) peptide, treated with 10 ng/mL TGF-β, or treated with the combination of peptide and TGF-β for 24 hours. Total RNA was extracted using the Qiagen miRNeasy kit (Qiagen) and treated with DNase1, according to the manufacturer's instructions. RNA was then quantified and converted to cDNA using the Clontech RNA to cDNA EcoDry Double Primed Premix (Takara). qPCR was performed using PowerUp Sybr Green (Invitrogen) on an Applied Biosystems Quant Studio 3 instrument. Primers were from IDT and were designed using IDT Primer Quest and NCBI Primer Blast tools. The delta-delta Ct method was used to determine relative expression of the pro-fibrotic mRNAs for tenascin C (TNC), collagen 1 (COL1A1), and fibronectin (FN1). GAPDH was used as a housekeeping gene. The results are shown in FIGS. 1A-H.

When cells were treated with a CXCL10 mimic peptide (IP-10-1, IP-10-2 or IP-10-3) in combination with TGF-β, the CXCL10 mimic peptides significantly reduced or prevented expression of TNC, COL1A1, and FN1, relative to treatment with TGF-β alone.

### Example 4: In Vivo Efficacy Studies

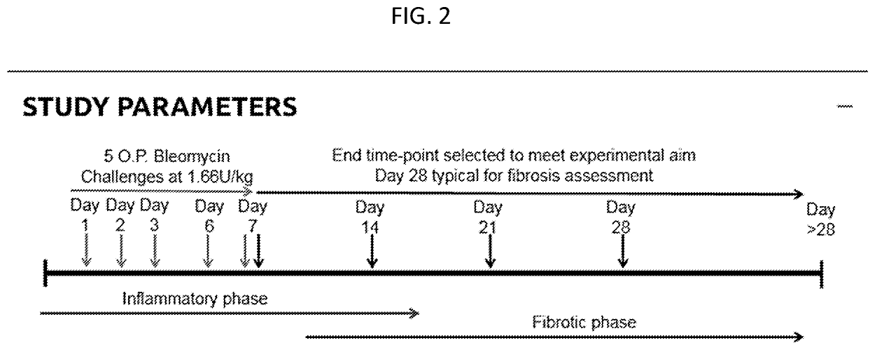

In vivo studies are performed using an accepted animal model of bleomycin exposure to induce lung fibrosis in a mouse. This animal model, which is the gold-standard in the field, was used to demonstrate initial efficacy for currently approved drugs, and has been endorsed by the American Thoracic Society (Kakugawa et al., Eur Respir J 24(1): 57-65, 2004; Wollin et al., J Pharmacol Exp Ther 349(2): 209-220, 2014; 2014, Jenkins et al.,Am J Respir Cell Mol Biol 56(5): 667-679, 2017). Peptides (one or more of the CXCL10 peptide mimics) are administered between weeks 2-3 of bleomycin administration and lung fibrosis is assessed via histological examination and immunofluorescence. This animal model uses wild-type, non-aged mice and the experimental protocol is 14-21 days long. A schematic of an exemplary protocol is shown in FIG. 2.

### Example 5: Aerosolization of CXCL10 Mimic Peptides

This example describes evaluation of different nebulizer systems to identify nebulizers that can efficiently deliver aerosolized CXCL10 mimic peptides to the deep lung.

All studies were performed with a Next Generation Impactor. This device separates an aerosol into 8 different size classes between 0.98 and 14 μm. The size of an aerosol affects where and if it will deposit in the lung when it is inhaled. A size of about 1 to about 5 μm is generally most effective. Aerosols larger than 5 μm will penetrate poorly in the lung and may deposit in the mouth or throat. Aerosols much smaller than 1 μm may be inhaled and exhaled without depositing. This impactor study was performed to determine how much of the active peptide is found in collection bins associated with “respirable aerosols.” Typically, bins 3-7 are associated with the size range of 0.98-5.39 μm.

Peptide solutions of 100 ng/ml and 500 ng/ml were aerosolized using: (1) an AEROECLIPSE™ BAN nebulizer with Ombra Compressor and collected aerosol with the impactor; and (2) an AEROGEN® Ultra vibrating mesh nebulizer. A 100 ng/ml peptide solution was also aerosolized in an AirLife Sidestream nebulizer run with a DeVilbiss 8650D compressor set to 30 L/min and collected aerosol. The collection stages were washed with 5 ml of DI water and stored.

Peptide content in the samples is measured by mass spectrometry, or another suitable method. Analysis of the samples enables determination of a Mass Median Aerodynamic Diameter (MMAD) associated with each concentration and fine particle fraction (FPF). FPF is the portion of aerosolized peptide mass found in the impactor bins 3-7. If the FPF is high enough, this indicates that the nebulizer system tested can efficiently deliver peptide to the deep lung. In some cases, the FPG is at least 50%, at least 60%, at least 70%, at least 80% or at least 90%.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A method of treating or inhibiting the development of fibrosis in a subject, comprising administering to the subject by inhalation a therapeutically effective amount of a CXCL10 mimic peptide, or a therapeutically effective amount of a composition comprising a CXCL10 mimic peptide, wherein the peptide or composition is administered as an aerosol, thereby treating or inhibiting the development of fibrosis in the subject.

2. The method of claim 1, wherein the CXCL10 mimic peptide is 12 to 30 amino acids in length.

3. The method of claim 1, wherein the amino acid sequence of the CXCL10 mimic peptide is at least 90% identical to any one of SEQ ID NOs: 1-42.

4. The method of claim 3, wherein the amino acid sequence of the CXCL10 mimic peptide is at least 95% identical to any one of SEQ ID NOs: 1-42.

5. The method of claim 4, wherein the amino acid sequence of the CXCL10 mimic peptide comprises or consists of any one of SEQ ID NOs: 1-42.

6. The method of claim 1, wherein the composition comprises at least two, at least three, at least four or at least five different CXCL10 mimic peptides.

7. The method of claim 1, wherein the composition further comprises a pharmaceutically acceptable carrier.

8. The method of claim 1, wherein the aerosol droplets are between 1 and 5 μm in diameter.

9. The method of claim 1, wherein the peptide or composition is administered using a nebulizer, a dry powder inhaler or a metered dose inhaler.

10. The method of claim 1, wherein the composition comprises about 100 ng/ml to about 500 ng/ml of the peptide.

11. The method of claim 1, wherein the fibrosis is fibrosis of lung, liver, kidney, heart or skin of the subject.

12. The method of claim 11, wherein the subject suffers from idiopathic pulmonary fibrosis (IPF).


SEQUENCE LISTING (42 sequences)

SEQ ID NO: 1 - protein, 22 aa, Artificial Sequence
  Synthetic peptide
PESKAIKNLLKAVSKEMSKRSP

SEQ ID NO: 2 - protein, 22 aa, Artificial Sequence
  Synthetic peptide
PESKVIKNLLKVASKERSKRSP

SEQ ID NO: 3 - protein, 22 aa, Artificial Sequence
  Synthetic peptide
PESRVIKNLLRVVSKEKSRRSP

SEQ ID NO: 4 - protein, 16 aa, Artificial Sequence
  Synthetic peptide
FAKAIKNFAKAVAKFA

SEQ ID NO: 5 - protein, 16 aa, Artificial Sequence
  Synthetic peptide
VRSTSISGGSISTSRV

SEQ ID NO: 6 - protein, 17 aa, Artificial Sequence
  Synthetic peptide
IIPASQFGGFPQSAPII

SEQ ID NO: 7 - protein, 16 aa, Artificial Sequence
  Synthetic peptide
FSPRVEIGGIEVRPSF

SEQ ID NO: 8 - protein, 12 aa, Artificial Sequence
  Synthetic peptide
FPMFKGGKFMPF

SEQ ID NO: 9 - protein, 13 aa, Artificial Sequence
  Synthetic peptide
RLSLSIGGISLSR

SEQ ID NO: 10 - protein, 17 aa, Artificial Sequence
  Synthetic peptide
DDHKEINAGGANIEKHD

SEQ ID NO: 11 - protein, 13 aa, Artificial Sequence
  Synthetic peptide
DDHKEIGGIEKHD

SEQ ID NO: 12 - protein, 16 aa, Artificial Sequence
  Synthetic peptide
FSPHVEIGGIEVHPSF

SEQ ID NO: 13 - protein, 16 aa, Artificial Sequence
  Synthetic peptide
FTPHVEIGGIEVHPTF

SEQ ID NO: 14 - protein, 14 aa, Artificial Sequence
  Synthetic peptide
FTHVEIGGIEVHTF

SEQ ID NO: 15 - protein, 16 aa, Artificial Sequence
  Synthetic peptide
SISTSRVGGVRSTSIS

SEQ ID NO: 16 - protein, 16 aa, Artificial Sequence
  Synthetic peptide
FPQSAPIGGIIPASQF

SEQ ID NO: 17 - protein, 16 aa, Artificial Sequence
  Synthetic peptide
IEVRPSFGGFSPRVEI

SEQ ID NO: 18 - protein, 12 aa, Artificial Sequence
  Synthetic peptide
KFMPFGGFPMFK

SEQ ID NO: 19 - protein, 13 aa, Artificial Sequence
  Synthetic peptide
ISLSRGGRLSLSI

SEQ ID NO: 20 - protein, 17 aa, Artificial Sequence
  Synthetic peptide
ANIEKHDGGDDHKEINA

SEQ ID NO: 21 - protein, 16 aa, Artificial Sequence
  Synthetic peptide
IEVHPSFGGFSPHVEI

SEQ ID NO: 22 - protein, 16 aa, Artificial Sequence
  Synthetic peptide
IEVHPTFGGFTPHVEI

SEQ ID NO: 23 - protein, 14 aa, Artificial Sequence
  Synthetic peptide
IEVHTFGGFTHVEI

SEQ ID NO: 24 - protein, 13 aa, Artificial Sequence
  Synthetic peptide
EKHDIGGDDHKEI

SEQ ID NO: 25 - protein, 19 aa, Artificial Sequence
  Synthetic peptide
DRFNSFRQPGGQRFSNFRD

SEQ ID NO: 26 - protein, 20 aa, Artificial Sequence
  Synthetic peptide
FVWQVAARVGGVRAAVQWVF

SEQ ID NO: 27 - protein, 20 aa, Artificial Sequence
  Synthetic peptide
DGVQPFNYQGGQYNFPQVGD

SEQ ID NO: 28 - protein, 25 aa, Artificial Sequence
  Synthetic peptide
NDALAGLRMLIRGGRIMRLGALADN

SEQ ID NO: 29 - protein, 14 aa, Artificial Sequence
  Synthetic peptide
LRMLIRGGRILMRL

SEQ ID NO: 30 - protein, 19 aa, Artificial Sequence
  Synthetic peptide
DRFNSYRQPGGQRYSNFRD

SEQ ID NO: 31 - protein, 19 aa, Artificial Sequence
  Synthetic peptide
DRYNSYRQPGGQRYSNYRD

SEQ ID NO: 32 - protein, 20 aa, Artificial Sequence
  Synthetic peptide
FVWQVFARVGGVRAFVQWVF

SEQ ID NO: 33 - protein, 16 aa, Artificial Sequence
  Synthetic peptide
FVWFARYGGYRAFWVF

SEQ ID NO: 34 - protein, 20 aa, Artificial Sequence
  Synthetic peptide
DGFQPFNYQGGQYNFPQFGD

SEQ ID NO: 35 - protein, 25 aa, Artificial Sequence
  Synthetic peptide
NDALFGLRMLIRGGRIMRLGFLADN

SEQ ID NO: 36 - protein, 21 aa, Artificial Sequence
  Synthetic peptide
NDWLFRMLIRGGRIMRFLWDN

SEQ ID NO: 37 - protein, 17 aa, Artificial Sequence
  Synthetic peptide
WLFRMWIRGGRWMRFLW

SEQ ID NO: 38 - protein, 14 aa, Artificial Sequence
  Synthetic peptide
LRFLIRGGRILFRL

SEQ ID NO: 39 - protein, 14 aa, Artificial Sequence
  Synthetic peptide
LRFLFRGGRFLFRL

SEQ ID NO: 40 - protein, 14 aa, Artificial Sequence
  Synthetic peptide
FRFLFRGGRFLFRF

SEQ ID NO: 41 - protein, 14 aa, Artificial Sequence
  Synthetic peptide
FRWLFRGGRFLWRF

SEQ ID NO: 42 - protein, 14 aa, Artificial Sequence
  Synthetic peptide
FRWLWRGGRWLWRF